(19) United States
(12) Patent Application Publication
(10) Pub. No.: US 2019/0167386 A1
(43) Pub. Date: Jun. 6, 2019

(54) SMART ORTHODONTIC BRACKET

(71) Applicants: Sreevatsan RAGHAVAN, Noida (IN); Praful JAIN, Ghaziabad (IN)

(72) Inventors: Sreevatsan RAGHAVAN, Noida (IN); Praful JAIN, Ghaziabad (IN)

(21) Appl. No.: 16/268,539

(22) Filed: Feb. 6, 2019

Related U.S. Application Data
(63) Continuation-in-part of application No. 15/544,674, filed on Jul. 19, 2017, now abandoned.

(51) Int. Cl. A61C 7/14; A61C 7/16; A61C 7/00; A61C 19/04; A61B 5/1455; G01L 1/16; G01N 17/04

(52) U.S. Cl. CPC A61C 7/146; A61C 7/16; A61C 7/002; A61C 2204/005; A61B 5/14552; G01L 1/16; G01N 17/04; A61C 19/04

(57) ABSTRACT

The present invention provides an orthodontic bracket system with at least one 3D piezoelectric sensor and at least one detachable RFID frequency transmitter tag for determining even the slightest change in initial setup of orthodontic bracket system and further notifying the subscriber electronically via laser scanner. The orthodontic bracket assembly comprises of orthodontic bracket for holding the tooth; at least one arch wire for connecting the orthodontic brackets; and at least one piezoelectric sensor. The removable pulse oximeter is placed at the periphery of the bracket to assess the pulp vitality. At least one detachable wireless frequency transmitter RFID tag on the tie-wing permits clinical recycling of the bracket and system allows an atomic absorption photo spectrometer to determine the corrosion characteristics of arch wire and further allows a handheld laser scanner to determine the centre of resistance and moment of force of tooth.

### CROSS REFERENCE TO RELATED APPLICATIONS

This application is a continuation-in-part of U.S. patent application Ser. No. 15/544,674 filed on Jul. 19, 2017, each hereby incorporated by reference herein. The U.S. Ser. No. 15/544,674 the United States National Stage of International Patent Corporation Treaty application no. PCT/IB 2016/050272, filed Jan. 20, 2016 claiming priority of Indian Application No. 174/DEL/2015 dated Jan. 20, 2015.

### FIELD OF THE INVENTION

The present invention relates to a system for monitoring the variable factors of an orthodontic bracket. More particularly, the invention provides an orthodontic bracket assembly with a detachable wireless RFID frequency transmitter

### BACKGROUND OF THE INVENTION

Teeth are attached to the bone with little mini rubber band type fibres known as periodontal ligaments. These ligaments allow very small natural movements of the teeth while eating or using the teeth. This ligament provides a little space between the tooth and the bone known as the periodontal ligament space. Now, suppose some pressure is applied on the tooth, pushing it in one direction. This pressure either stretches (tension) the ligaments on one side of the tooth, or squishes (compresses) the ligaments on the other side of the tooth. Therefore, the tooth experiences a tension side and a compression sides. When the body senses these prolonged forces, it starts adding bone, with cells called osteoblasts on the tension side of the body. On the compression side, the body starts eating away bone with cells called osteoblasts. This system of removing bone on one side, while adding bone on the other side, is what allows teeth to move through bone. If the force is too large the system will not be very efficient. A light, continuous force is the most efficient way to move teeth. Therefore, when we put on braces, we are setting up a mechanical system to deliver forces to the teeth.

Orthodontics is a specific field of dentistry that diagnoses, prevents and treats irregularities of the teeth and face. Orthodontic treatment regarding irregularities of the teeth involves straightening of teeth, correcting an irregularity in bite, closing of unsightly gaps and bringing teeth and lips into proper alignment. Majorly, there are two types of braces i.e. removable and permanent. Removable braces can be put on or removed by patients and is custom made to fit the shape of each patient's mouth. Permanent braces such as metallic braces are the traditional braces with a system of very visible brackets and arch wires that requires specialized help for installation and removal. These metallic braces are cheap, offer more control over the desired tooth movements and therefore more popular. Although, the type of braces may vary but the goal of the treatment is the same i.e. to gradually move the teeth into the desired position.

With the exception of Invisalign brace, all other types of braces employ brackets on each and every tooth and connect them with a wire to rectify the irregularities. A dentist shall repeat the process many times over the period to set and align. A patient shall have to visit a dentist many times just to maintain a constant pressure to gradually move the teeth.

One of the major drawbacks associated with use of braces for rectification, is frequent pain. Constant adjustments are done to maintain force on a tooth so that irregularities are treated. Although, after a while when the tooth moves under applied force, the pressure slackens; so to maintain pressure constant adjustments are done. Therefore, each time a person undergoes check-up; orthodontist makes adjustments and bends, adjusts, tightens, or replaces the wire with a thicker wire to force your teeth to shift. The orthodontist may further change the ties used to hold the arch wire to the brackets and make any other necessary adjustments to your braces. Each adjustment is a step toward straight teeth, but the movement of teeth is painful. Hence each time a person undergoes the check-up, the orthodontist determines the magnitude of force applied by braces. The prior art presented here provide modern brackets to adjust the magnitude of force so that the arch wire does not slacken and relay such messages to a central processor to make adjustments, timely.

In WO2007133422A3, the invention calculates, force magnitudes and/or directions may be determined objectively using orthodontic brackets having an elastomeric member which allows one portion of the bracket to be resiliently moveable relative to at least on other portion of the bracket. In a preferred embodiment, the brackets include a lower base member, an upper bracket member, and an elastomeric layer interposed between the lower base and upper bracket members. The orthodontic bracket is advantageously employed as part of a system whereby the orthodontic bracket includes an elastomeric member which allows at least one portion of the bracket to be resiliently movable relative to at least one other portion of the bracket in response to an applied force, and at least one force-responsive sensor operatively associated with the orthodontic bracket for generating a detectable signal in response to movement of the at least one and other portions of the bracket. A detector is provided so as to allow for the wireless detection of the force-responsive signal generated by the force-responsive sensor and issue an output signal in response thereto. A processor receives the output signal from the detector to provide an indication of magnitude and/or direction of the force applied to the orthodontic bracket.

In EP1505921B1, the invention relates to an orthodontic bracket (B) serving to fix a tight-fitting orthodontic appliance to a tooth, comprising a bracket base (2), which is to be fixed to a tooth (1), and a bracket attachment (3) for attaching a force/pressure and/or torque application device. According to the invention, at least one sensor device is provided between the bracket base (2) and the bracket attachment (3) for measuring a force, which is imparted by the bracket attachment (3) to the bracket base (2), an imparted pressure and/or an imparted torque. The invention also relates to a tight-fitting orthodontic appliance comprising at least one orthodontic bracket of the aforementioned type. The sensor device (4) integrated inside bracket (B) makes it possible to measure the forces, pressures and/or torques that are actually acting on the tooth (1) thereby enabling the orthodontist to take this data into account during the use of the orthodontic appliance. The invention also relates to a removable orthodontic appliance.

However, these closest prior art provide modern brackets to adjust the magnitude of force so that the arch wire does not slacken but there are no prior arts that accounts for the pain and hence adjust the arch wire.

Due to lack in technology, there are prefixed adjustments that do not account for the amount of pain suffered by each patient's. As each person is different due to the unique bone physiology and responds differently to the same stress levels of the orthodontic appliance. Most of the time a patient's ability to withstand pain is the only parameter for such adjustments. Accordingly, there is a need for a solution to overcome the shortcomings in the state of the art. Therefore, there is requirement for the brackets apply force within the biological limits for internal pain due to the pain caused by changes in blood flow that occur when the braces apply pressure to the teeth.

Further, the entire prior art installs their RFID tags on the bracket base, which is removed with a blow torch that damages the wireless transmitter, hence they are not recyclable.

Further the prior art, the two bracket members are connected by an elastomeric component; therefore there is force decay of the elastomeric component in saliva causing improper force level detection. Also no prior art mentions frictional force detection and many place the piezo sensor in the bracket-slot which increases friction and none also measure the integrity of the bonding of the bracket to the teeth. The present invention removes all the drawbacks of the prior art.

### OBJECT OF THE INVENTION

Accordingly, the main object of the invention is to provide a system for monitoring the variable factors of an orthodontic bracket over a period of time.

Another main object of the invention is to provide a method for communicating the variable factors of orthodontic bracket to the central processing system for determining even the slightest change in the setup of orthodontic bracket assembly such as but not limiting to lose wire(s), broken bracket(s), conformational or spatial change, change in pressure, strain or force etc.; and notifying the dentist and/or user electronically about the change.

Yet another object of the invention is to provide an orthodontic bracket assembly fitted with at least one detachable RFID tag.

Yet another object of the invention is to assess pulp vitality using a removable pulse oximeter placed at the periphery of the orthodontic bracket.

Yet another object of the invention is to configure a photo spectrometer to determine the corrosion characteristics of the orthodontic arch wires.

Yet another object of the invention is to configure a handheld laser scanner to read the data from the RFID tag and communicate to central processing system.

Yet another object of the invention is to provide a handheld laser scanner configured to determine the centre of resistance of a tooth.

Yet another object of the invention is to provide a detachable wireless frequency transmitter RFID tag to anywhere on the tie-wing of the bracket, configured to permit clinical recycling of the bracket which gets debonded or inaccurately positioned and require repositioning of the brackets.

Still another object of the invention is to make the orthodontic bracket assembly compatible with other remotely/hand held devices.

### SUMMARY OF THE INVENTION

The present invention relates to a system for monitoring the variable factors of an orthodontic bracket. More particularly, the invention provides an orthodontic bracket assembly with a detachable wireless RFID frequency transmitter.

In a preferred embodiment of the present invention is provided an orthodontic bracket assembly comprising a plurality of orthodontic bracket for holding teeth, a plurality of orthodontic bracket base glued on teeth, at least one arch wire for connecting the plurality of orthodontic bracket and a plurality of tie wings of orthodontic bracket. The plurality of orthodontic bracket base is installed with at least one 3D piezoelectric sensor to sense change in mechanical force exerted by the orthodontic bracket on teeth and produce a charge. The plurality of tie wings of the orthodontic bracket is installed with at least one detachable RFID tag to receive the charge produced by said 3D piezoelectric sensor and convert it into a machine readable data to transmit to a central processing system. The 3D piezoelectric sensor and said detachable RFID tag are connected by a relay wire and at least one pin and socket joint.

In another embodiment of the present invention is provided a system for monitoring the variable factors comprising an orthodontic bracket assembly, a pulse oximeter, a laser scanner, a photo spectrometer, a central processing system comprising of a central processor and a visual display unit. The variable factor includes but not limited to position, pulp vitality, corrosion characteristics of the orthodontic bracket assembly, change in mechanical force on teeth. The orthodontic bracket assembly comprises of a bracket base installed with at least one 3D piezoelectric sensor, an arch wire for connecting a plurality of orthodontic brackets and a tie wing and at least one detachable RFID tag for determining change in mechanical force. The pulse oximeter is attached to periphery of said orthodontic bracket assembly for determining the pulp vitality. The photo spectrometer determines the corrosion characteristics of said arch wires of said orthodontic bracket assembly. The laser scanner scans the teeth at a height to determine the position of teeth. The variable factors are communicated to the central processing system to give an indication for clinical recycling to a clinician.

Still another embodiment of the present invention, the invention provides at least one detachable wireless frequency RFID tag to be placed anywhere on the tie-wing of the bracket to permit clinical recycling of the bracket which gets debonded or inaccurately positioned and require repositioning of the brackets. Generally while clinical recycling of the brackets, the bracket base is heated with a blow torch to remove the adhesive from the bracket base so that bracket can be bonded again to tooth. A wireless transmitter attached to bracket base gets damaged while this clinical recycling is performed. Therefore, the present invention provides a detachable wireless frequency transmitter (RFID) on the bracket of tie-wing.

### BRIEF DESCRIPTION OF DRAWINGS

A complete understanding of the device and method of the present invention may be obtained by reference to the following drawing:

FIG. 1 shows an orthodontic bracket in accordance to the present invention.

FIG. 2 shows the orthodontic bracket showing the top view of the base in accordance to the present invention.

FIG. 3 shows the orthodontic bracket showing the bottom view of the bracket.

FIG. 4 shows a handheld laser scanner connected to monitor in accordance to the present invention.

FIG. 5 shows a pulse oximeter to assess the pulp vitality in accordance to the present invention.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention relates to a system for monitoring the variable factors of an orthodontic bracket. More particularly, the invention provides an orthodontic bracket assembly with a detachable wireless RFID frequency transmitter.

In preferred embodiment of the present invention is provided an orthodontic bracket assembly comprising a plurality of orthodontic bracket for holding teeth, a plurality of orthodontic bracket base to attach on teeth, at least one arch wire for connecting the plurality of orthodontic bracket and a plurality of tie wings of orthodontic bracket. The plurality of orthodontic bracket base is installed with at least one 3D piezoelectric sensor to sense change in mechanical force exerted by the orthodontic bracket on teeth and produce a charge. The plurality of tie wings of the orthodontic bracket is installed with at least one detachable RFID tag to receive the charge produced by said 3D piezoelectric sensor and convert it into a machine readable data to transmit to a central processing system. The 3D piezoelectric sensor and said detachable RFID tag are connected by a relay wire and at least one pin and socket joint.

In another embodiment of the present invention is provided a system for monitoring the variable factors comprising an orthodontic bracket assembly, a pulse oximeter, a laser scanner, a photo spectrometer, a central processing system comprising of a central processor and a visual display unit. The variable factor includes but not limited to position, pulp vitality, corrosion characteristics of the orthodontic bracket assembly, change in mechanical force on teeth. The orthodontic bracket assembly comprises of a bracket base installed with at least one 3D piezoelectric sensor, an arch wire for connecting a plurality of orthodontic brackets and a tie wing and at least one detachable RFID tag for determining change in mechanical force. The pulse oximeter is attached to periphery of said orthodontic bracket assembly for determining the pulp vitality. The photo spectrometer determines the corrosion characteristics of said arch wires of said orthodontic bracket assembly. The laser scanner scans the teeth at a height to determine the position of teeth. The variable factors are communicated to the central processing system to give an indication for clinical recycling to a clinician.

Still another embodiment of the present invention, the invention provides at least one detachable wireless frequency RFID tag to be placed anywhere on the tie-wing of the bracket to permit clinical recycling of the bracket which gets debonded or inaccurately positioned and require repositioning of the brackets. Generally while clinical recycling of the brackets, the bracket base is heated with a blow torch to remove the adhesive from the bracket base so that bracket can be bonded again to tooth. A wireless transmitter attached to bracket base gets damaged while this clinical recycling is performed. Therefore, the present invention provides a detachable wireless frequency transmitter (RFID) on the bracket of tie-wing.

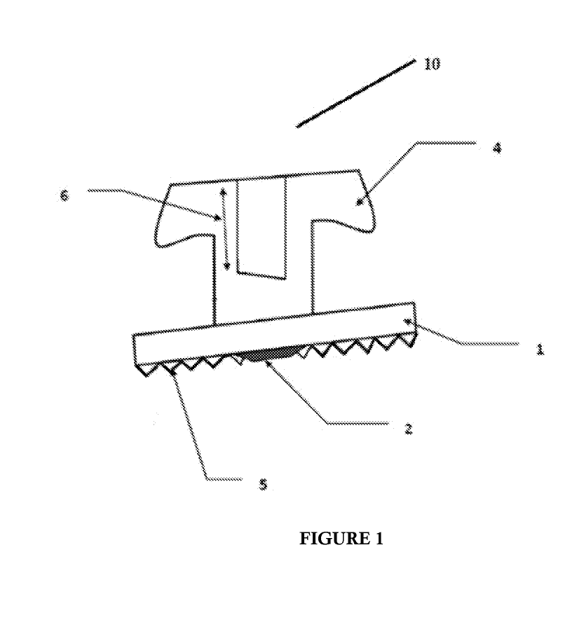

Now referring to FIG. 1, an orthodontic bracket 10 is shown according to an embodiment of the present invention. A bracket base 1 is shown to have specially designed multiple grooves 5. The bracket base 1 is attached by means of an adhesive and rests on the teeth due to traction provided by multiple grooves 5. A 3D piezoelectric sensor 2 is welded on the grooves of the bracket base 1. A tie wing 4 of the orthodontic bracket is installed with a detachable wireless RFID frequency transmitter 8. A removable pulse oximeter is attached to the orthodontic bracket at a height 6 to diagnose the pulp vitality during fitment of bracket on a subject's denture, and during maintenance clinical recycling.

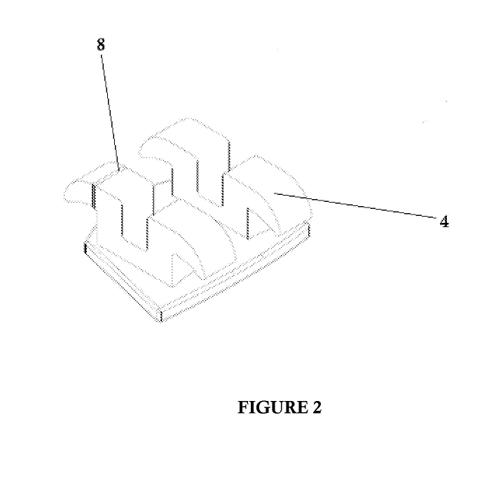

Now referring to FIG. 2, a top view of the orthodontic bracket according to the present invention is shown. The detachable RFID tag 8 is installed on the tie wing 4 of the orthodontic bracket. The RFID tag has an analog to digital converter, a micro processor, a memory and an antenna which is adapted to measure the size of electrical charge received from a 3D piezoelectric sensor which senses the change in mechanical force applied by bracket on tooth in three planes of spaces. Said electrical charge activates the microprocessor and analog to digital converter to convert the electric charge into a machine readable data and store in the memory and the stored data is transferred to the central processing system when scanned through a laser scanner. The central processing system comprises of a central processor and a visual display unit. The central processing system gives an indication for clinical recycling to a clinician. The change in mechanical force corresponds to the tension on arch wires, pressure exerted on individual tooth, alignment of tooth such as crowding or spacing, broken arch wire, loose arch wire.

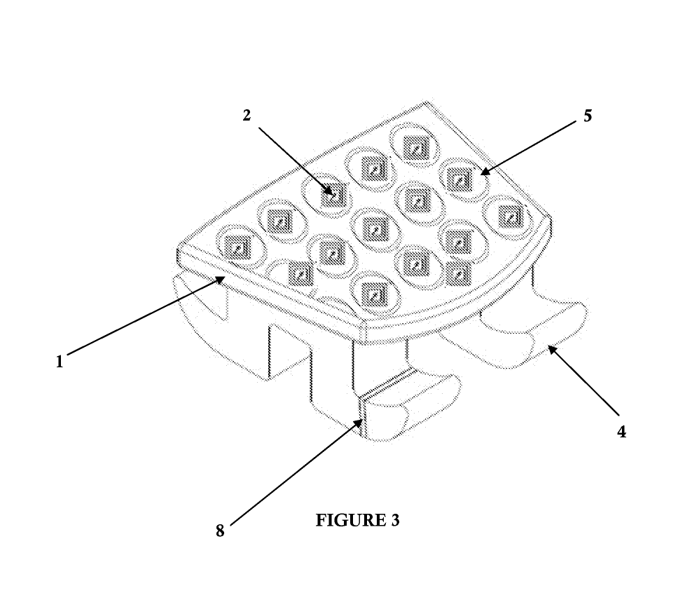

Now referring to FIG. 3, a bottom view of orthodontic bracket is shown having base 1 and grooves 5. The piezoelectric sensor 2 is welded on the grooves 5 of base 1. The bottom view elucidates the detachable wireless frequency transmitter 8 such as RFID installed on the tie wings 4. The 3D piezoelectric sensor and said detachable RFID tag are connected by a relay wire and at least one pin and socket joint.

In one of the arrangements, the 3D piezoelectric sensor is having relay wires soldered to it and there is a pin joint connected to the end of these relay wires and socket joint is provided in said detachable RFID tag to be connected when in use and snapped during clinical recycling. In another arrangement, the 3D piezoelectric sensor is having a pin/socket joint soldered to it and on the other hand a pin/socket joint is soldered to the RFID tag and the relay wires have corresponding pin/socket joint for establishing connection.

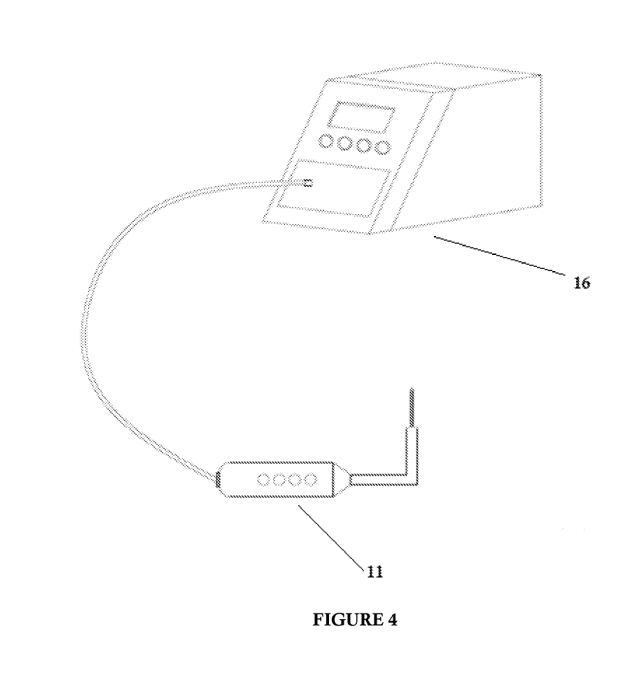

Now referring to FIG. 4, a handheld laser scanner is shown to measure the centre of resistance of tooth when intrusive forces are applied to them. The location of the centre of resistance (CR) is considered as an essential factor in the planning of orthodontic tooth movements. With exact knowledge of the CR, the force system that must be applied to the crown of the tooth to achieve the desired tooth movement can be determined. The laser scanner is augmented with a probe 11 to determine the exact amount of centre of resistance required for tooth retraction and also to determine the moment of force. The centre of resistance and moment of force are then displayed on a visual display unit 16. The varying levels of forces such as 50 g, 100 g and 200 g are applied to different splints holding parts of teeth such as splints between the central incisors, between central and lateral incisors, between lateral incisors and canines, between the canines and first premolars. The metal splints are used to hold the anterior teeth to minimize the individual tooth movements. An argon laser is directed to the metal splint and reflected beam is interpreted by microscope and speckle is recorded. A well recognizable speckle is positioned at the centre of the hairline while adjusting the objective of microscope. The splint is loaded and amount of displacement of the reflected speckle are noted. An upward or downward movement of the speckle indicates a counter clockwise and clockwise rotation of anterior.

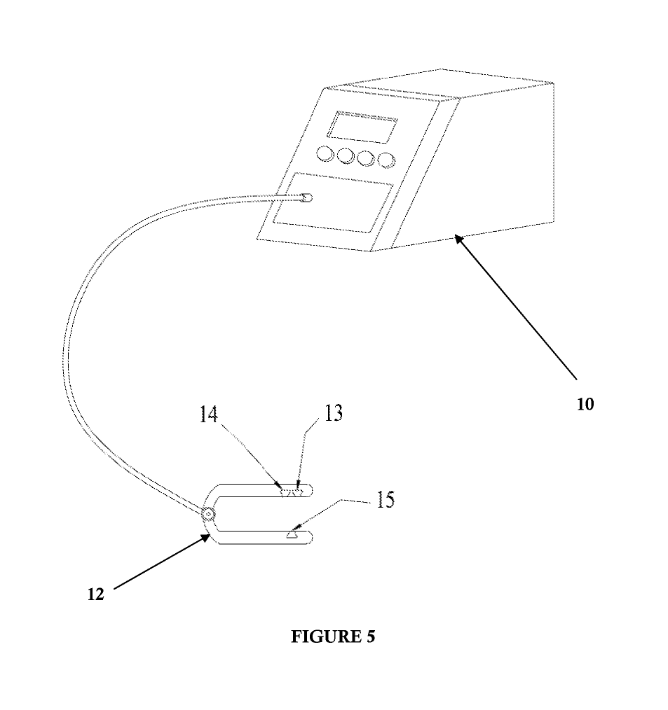

Now referring to FIG. 5, a pulse oximeter is shown. As known in the prior art, a pulse oximeter uses a probe containing two light-emitting diodes (LEDs) out of which one LED transmits red light (660 nm) 13 and the other transmits infrared light (900-940 nm) 14 to measure the absorption of oxygenated and deoxygenated hemoglobin, respectively (it operates at 500 on/off cycles/s). Oxygenated and deoxygenated hemoglobin absorb different amounts of red and infrared light. This light is received by a photo detector diode 15. The probe is connected from the pulse oximeter sensor 12 to the pulse oximeter monitor 10. The pulsatile change in the blood volume causes periodic changes in the amount of red and infrared light absorbed by the vascular bed before reaching the detector. The relationship between the pulsatile change in the absorption of red light and infrared light is assessed by the oximeter showing the saturation of arterial blood. It uses this information to determine the oxygen saturation levels. By monitoring changes in oxygen saturation, pulse oximeter is able to detect pulpal inflammation or partial necrosis in teeth. The critical requirement of using pulse oximeter in dentistry is as follows: sensor should conform to the size, shape, and anatomical contours of teeth, light-emitting diode sensor and the photoreceptor should be as parallel as possible to each other so that the photoreceptor sensor receives the light-emitted from LED, the sensor holder should allow firm placement of the sensor onto the tooth to obtain accurate measurements. The pulse oximeter is used for analysing the different pulpal conditions of the test tooth such as healthy pulp (PC), reversible pulpitis, irreversible pulpitis, pulpal necrosis.

The orthodontic bracket assembly allows atomic absorption photo spectrometer to determine the corrosion characteristics of as received and grinded arch wires. The corrosion behaviour of the arch wires determines their biocompatibility. The general test of atomic absorption spectro photometer to determine the corrosion characteristics of the arch wire as known in the prior art uses different orthodontic wires of nominal compositions such as nickel-titanium, titanium-molybdenum, cobalt-krypton and stainless steel wires. These arch wires are tested either as as-received form or grinded form. These forms are totally immersed in NaCl solution. After one week, the specimens are removed from the solution and rinsed with deionized water and again immersed in a freshly prepared NaCl solution and finally removed after four weeks. Nitric acid is added to the NaCl solution to prevent adsorption of dissolved metallic ions. The amount of metallic ions released into the solution is determined by spectro photometer. The means and standard deviation of the amount of metallic ions are calculated. As observed, the corrosion rate of the wires decreased with the passage of immersion time in NaCl solution. The amount of metal ions released from most of the alloys are larger than the as-received wires. The amount of metallic ions released by as received wires is 2000 ng-cm²

We claim:

1. An orthodontic bracket assembly comprising of:
a plurality of orthodontic bracket for holding teeth, said orthodontic bracket comprising a base glued to the teeth, a plurality of tie wings, and at least one arch wire interconnecting the plurality of orthodontic bracket;
at least one RFID tag; and
at least one 3D piezoelectric sensor;
wherein,
said at least one 3D piezoelectric sensor is placed at the base of the orthodontic bracket, said at least one 3D piezoelectric sensor senses a change in mechanical force exerted by the orthodontic bracket on teeth and produces a charge;
said at least one 3D piezoelectric sensor is in communication with said at least one RFID tag through a relay wire;
said at least one RFID tag is placed at the tie-wing of the orthodontic bracket to receive the charge produced by said 3D piezoelectric sensor and convert it into a machine readable data;
said at least one RFID tag is detachable.

2. The orthodontic bracket assembly as claimed in claim 1, wherein said 3D piezoelectric sensor is welded on said orthodontic bracket base.

3. The orthodontic bracket assembly as claimed in claim 1, wherein said at least one RFID tag is connected to the at least one 3D piezoelectric sensor by at least one pin and socket joint.

4. The orthodontic bracket assembly as claimed in claim 1, wherein the change in mechanical force corresponds to tension on arch wires, pressure exerted on individual tooth, alignment of tooth such as crowding or spacing, broken arch wire, loose arch wire.

5. The orthodontic bracket assembly as claimed in claim 4, wherein said at least one RFID tag is detached during clinical recycling.

6. The orthodontic bracket assembly as claimed in claim 1, wherein the RFID tag comprises of a microprocessor, a memory, an analog to digital converter and an antenna.

7. An orthodontic bracket system for monitoring variable factors of orthodontic bracket comprising of:
an orthodontic bracket assembly comprising of:
a plurality of orthodontic bracket for holding teeth, said orthodontic bracket comprising a base glued to the teeth, a plurality of tie wings, and at least one arch wire interconnecting the plurality of orthodontic bracket;
at least one RFID tag; and
at least one 3D piezoelectric sensor;
a removable pulse oximeter;
a laser scanner;
a photo spectrometer;
a central processing system comprising of a central processor and a visual display unit;
wherein,
variable factors of orthodontic bracket include but not limited to position, pulp vitality, corrosion characteristics of the orthodontic bracket assembly, change in mechanical force on teeth;
said at least one 3D piezoelectric sensor is placed at the base of the orthodontic bracket, said at least one 3D piezoelectric sensor senses change in mechanical force exerted by the orthodontic bracket on teeth and produces a charge;
said at least one RFID tag is placed at the tie-wing of the orthodontic bracket to receive the charge produced by said 3D piezoelectric sensor and convert it into a machine readable data;
said removable pulse oximeter is attached to periphery of said orthodontic bracket for determining the pulp vitality;
said photo spectrometer determines the corrosion characteristics of said arch wires of said orthodontic bracket assembly;
said laser scanner scans the teeth at a height to determine the position of teeth; and
said variable factors of orthodontic bracket are communicated to the central processing system to give an indication for clinical recycling to a clinician.

8. The system for monitoring variable factors of orthodontic bracket as claimed in claim 7, wherein said removable pulse oximeter is attached at the periphery of said orthodontic bracket to assess different pulpal conditions such as healthy pulp (PC), reversible pulpitis, irreversible pulpitis and pulpal necrosis.

9. The system for monitoring the variable factors of orthodontic bracket as claimed in claim 7, wherein the position of teeth is determined by calculating shift in centre of resistance using a laser scanner.

10. The system for monitoring the variable factors of orthodontic bracket as claimed in claim 7, wherein the change in mechanical force corresponds to the tension on arch wires, pressure exerted on individual tooth, alignment of tooth such as crowding or spacing, broken arch wire, loose arch wire.

11. The system for monitoring the variable factors of orthodontic bracket as claimed in claim 7, wherein said at least one RFID tag is detached during clinical recycling.

12. The system for monitoring the variable factors of orthodontic bracket as claimed in claim 7, wherein the arch wires of the orthodontic bracket assembly are removed for determining the corrosion characteristics and used again if not corroded.

13. The system for monitoring the variable factors of orthodontic bracket as claimed in claim 7, wherein the machine readable data is scanned through a laser scanner and transmitted to central processing system to give an indication for clinical recycling to a clinician.
